(19) United States
(12) Patent Application Publication
(10) Pub. No.: US 2023/0414851 A1
(43) Pub. Date: Dec. 28, 2023

(54) DIALYSIS SYSTEM HAVING A DISINFECTION UNIT

(71) Applicants: BAXTER INTERNATIONAL INC., Deerfield, IL (US); BAXTER HEALTHCARE SA, Glattpark (Opfikon) (CH)

(72) Inventors: Oskar Erik Frode Styrbjorn Fallman, Lund (SE); Bjorn Ericson, Lund (SE)

(21) Appl. No.: 18/215,520

(22) Filed: Jun. 28, 2023

Related U.S. Application Data
(60) Provisional application No. 63/356,396, filed on Jun. 28, 2022.

(51) Int. Cl. A61M 1/28

(52) U.S. Cl. CPC A61M 1/282; A61M 2205/3331; A61M 2205/3368

(57) ABSTRACT

A peritoneal dialysis (“PD”) system includes a PD machine including a housing, a PD fluid pump housed by the housing, a plurality of PD fluid lines, and a plurality of PD fluid line connectors positioned and arranged at the housing to accept distal ends of the PD fluid lines to perform a disinfection sequence. The PD system also includes a disinfection unit including a disinfection unit housing, a PD fluid line connector positioned and arranged at the disinfection unit housing for receiving one of the PD fluid lines of the PD machine for the disinfection sequence, a line extending from the disinfection unit housing for connecting to one of the PD fluid line connectors of the PD machine for the disinfection sequence, and a disinfection fluid pump housed by the disinfection unit for pumping disinfection fluid during the disinfection sequence.

### PRIORITY CLAIM

This application claims priority to and the benefit as a non-provisional application of U.S. Provisional Patent Application No. 63/356,396, filed on Jun. 28, 2022, the entire contents of which are hereby incorporated by reference and relied upon.

### TECHNICAL FIELD

The present disclosure relates generally to medical fluid treatments, and in particular to dialysis fluid treatments that require fluid heating.

### BACKGROUND

Due to various causes, a person's renal system can fail. Renal failure produces several physiological derangements. For instance, it is no longer possible to balance water and minerals or to excrete daily metabolic load. Additionally, toxic end products of metabolism, such as urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins, and excess water to take place. The source may include multiple liters of dialysis fluid including several solution bags.

The PD machines pump used or spent dialysate from the patient's peritoneal cavity, though the catheter, to drain. As with the manual process, several drain, fill, and dwell cycles occur during dialysis. A “last fill” may occur at the end of an APD treatment. The last fill fluid may remain in the peritoneal chamber of the patient until the start of the next treatment, or may be manually emptied at some point during the day.

In any of the above modalities involving an automated machine, component wear may become an issue. As components are used more frequently, at higher flowrates or power inputs, and for longer periods of time, component wear increases. Because the automated machines provide critical treatment to their patients, it is important that the machines be as reliable as possible. When an automated machine goes down in a clinic, stress on the remaining machines is increased. When an automated machine goes down in a patient's home, a replacement machine needs to swap out the defective machine quickly so that the patient's treatment schedule is interrupted as little as possible.

For each of the above reasons, it is desirable to provide an APD machine that reduces component wear.

### SUMMARY

The present disclosure sets forth an automated peritoneal dialysis (“PD”) system, which provides one or more PD treatment improvement. The system includes a PD machine or cycler. The PD machine is capable of delivering fresh, heated PD fluid to a patient at, for example, 14 kPa (2.0 psig) or higher. The PD machine is capable of removing used PD fluid or effluent from the patient at, for example, between −5 kPa (−0.73 psig) and −15 kPa (−2.2 psig), such as −9 kPa (−1.3 psig) or higher. Fresh PD fluid may be delivered via a dual lumen patient line to the patient and is first heated to a body fluid temperature, e.g., 37° C. The heated PD fluid is then pumped through a fresh PD fluid lumen of the dual lumen patient line to a disposable filter set, which is connected to the patient's transfer set, which is in turn connected to an indwelling catheter leading into the patient's peritoneal cavity. The disposable filter set communicates fluidly with the fresh and used PD fluid lumens of the dual lumen patient line. The disposable filter set is provided in one embodiment as a last chance filter for the PD machine, which may be heat disinfected between treatments.

The system may include one or more PD fluid container or bag that supplies fresh PD fluid to the PD machine or cycler. The PD machine or cycler may include internal lines having two-way or three-way valves and at least one PD fluid pump for pumping fresh PD fluid from the one or more PD fluid container or bag to a patient and for removing used PD fluid from the patient to a house drain or drain container. One or more flexible PD fluid line leads from the PD machine or cylcer's internal lines to the one or more PD fluid container or bag. The flexible dual lumen patient line mentioned above leads from the PD machine or cylcer's internal lines to the patient. A flexible drain line leads from the PD machine or cylcer's internal lines to the house drain or drain container. The system in one embodiment disinfects all internal lines, the PD fluid lines and the dual lumen patient line after treatment for reuse in the next treatment.

The disinfection may involve heat disinfection using leftover fresh PD fluid, which may be heated to a disinfection temperature from about 70° C. to 90° C. or higher as discussed herein. To provide good disinfection and heat transfer to each of the lines disinfected, the PD fluid pump if used would need to run at a relatively high pumping rate, resulting in the risk of cavitation and the wear of the associated pump seals. It is accordingly contemplated not to use the PD fluid pump, either at all, or as the main disinfection fluid driving pump during the disinfection sequence.

The system of the present disclosure instead provides a disinfection unit, which may be separate from the PD machine or cycler. The disinfection unit includes an additional pump, which may be used in addition to or instead of the PD fluid pump during disinfection. To that end, the disinfection unit pump may be a sturdy pump that is less accurate than the PD fluid pump, but which may operate at higher flowrates than the PD fluid pump without incurring appreciable wear. The disinfection unit pump may for example be a centrifugal pump. The higher flowrate may for example be from 200 milliliters (“ml”)/min to 500 ml/min. The disinfection unit optionally includes a second, disinfection unit inline heater, which is selected specifically to heat fluid moving at the elevated disinfection flowrates and to heat the disinfection fluid to a temperature specified herein or to a potentially higher disinfection temperature, such as 90° C. to 110° C.

The disinfection unit in one embodiment includes a PD fluid line connector that accepts a distal end of any desired one of the reusable, flexible PD fluid lines of the PD fluid machine or cycler. The disinfection unit also includes a flexible line that extends to the open PD fluid line connector located at the PD fluid machine or cycler. The two fluid connections to the disinfection unit enable the internal lines and fluid components of the disinfection unit to form part of the PD machine or cycler's disinfection loop during the disinfection sequence. Sealed connections to the PD fluid line connector and the flexible line may be made by the patient or caregiver after treatment when it is time for disinfection.

Electrical control may be implemented in the disinfection unit in a variety of different ways. When only a disinfection unit pump is provided, then an on/off button or switch may be employed, which the patient or caregiver may switch to “on” to initiate a disinfection sequence, e.g., after confirming that the PD machine or cycler has been properly configured for disinfection. The disinfection unit may include an alternating current (“AC”) plug and whatever electronics are necessary to convert the AC voltage to the disinfection unit pump voltage if necessary. When a disinfection unit heater is also provided, an on/off switch may still be employed for the patient or caregiver to actuate. However, at least one temperature sensor and a controller for using an output from the one or more temperature sensor as feedback for controlling power to the heater is also provided so that the disinfection fluid, which may be PD fluid, exits the disinfection unit to the PD machine or cycler at a desired disinfection temperature.

When needed, the disinfection unit may also provide one or more pressure sensor and a controller for using an output from the one or more pressure sensor as feedback for controlling the disinfection unit pump, so that the disinfection fluid exits the disinfection unit to the PD machine or cycler at a desired disinfection pressure. The temperature sensor controller and the pressure sensor controller may be part of an overall disinfection unit control unit, which may include one or more processor and one or more memory. The disinfection unit may also include a user interface, e.g., a single or multi-line light-emitting diode (“LED”) or liquid crystal display (“LCD”), wherein, the disinfection unit control unit may further include a video controller.

As discussed herein, the control unit of the PD machine or cycler may also include a transceiver and a wired or wireless connection to a network (not illustrated), e.g., the internet, etc. The control unit of the disinfection unit may likewise include a transceiver for wireless communication with the control unit of the PD machine or cycler, e.g., via Bluetooth or Wifi wireless technologies. In an embodiment, instead of the patient or caregiver powering the disinfection unit at the time of disinfection, the control unit of the PD machine or cycler tells the control unit of the disinfection unit when to power the disinfection unit pump and the disinfection unit heater if provided, e.g., after the control unit of the PD machine or cycler has confirmed that the patient or caregiver has made all necessary connections for the disinfection sequence. The patient or caregiver here may accordingly connect the disinfection unit to the PD machine or cycler as described herein, setup the PD machine or cycler for disinfection, and then be free to leave and perform other tasks.

It is also contemplated to wirelessly transmit the outputs from one or more temperature sensor and/or one or more pressure sensor of the PD machine or cycler to the corresponding controllers of the control unit of the disinfection unit for use as feedback to control the power applied to the disinfection unit heater and/or the rate (or other pressure adjusting parameter) of the disinfection unit pump. In this manner, disinfection fluid, e.g., PD fluid, may be delivered from the disinfection unit at a desired disinfection temperature and/or pressure without having to provide corresponding temperature and/or pressure sensors within the unit.

During disinfection, the control unit of the PD machine or cycler is programmed in one embodiment to set the PD fluid pump, which may be a piston pump, such that it is in a free-flow position where the disinfection fluid may flow through the PD fluid pump without operating the pump. In an alternative embodiment, the control unit operates the PD fluid pump, e.g., at a slow or reduced speed during disinfection. Likewise, the control unit of the PD machine or cycler is programmed in one embodiment to not power the inline heater of the PD machine or cycler during disinfection if the disinfection unit is provided with its own heater. In an alternative embodiment, the control unit provides a low amount of power to the inline heater of the PD machine or cycler during disinfection even if the disinfection unit is provided with its own heater. The control unit provides full power to the inline heater of the PD machine or cycler during disinfection even if the disinfection unit is not provided with its own heater. Here, the inline heater of the PD machine or cycler is relied upon to heat the disinfection fluid to the desired disinfection temperature.

After disinfection, the disinfection unit may be left connected to the PD machine or cycler until it is time for the next treatment. At that time, the patient or caregiver may (i) disconnect the disinfection unit's flexible line from the PD fluid line connector located at the PD fluid machine or cycler, (ii) disconnect the distal end of the reusable, flexible PD fluid line of the PD fluid machine or cycler from the PD fluid line connector of the disinfection unit, and (iii) connect the disinfection unit's flexible line to the disinfection unit's PD fluid line connector. Such disconnection and reconnection closes the disinfection unit fluidically and maintains it in a relatively clean and disinfected state. The disinfection unit is then ready for its next use.

The advantages to the use of the disinfection unit include, but are not limited to, increased flowrate during the disinfection sequence. It is also contemplated that the reusable valves of the PD machine or cycler will not have to be opened and closed as much because the increased flowrate increase surface contact efficiency. The speed of the PD fluid pump may be reduced or even stopped. The risk of cavitation within the PD fluid pump is also lowered and perhaps eliminated, increasing disinfection efficiency and reducing wear on the PD fluid pump. Disinfection fluid temperatures may also be increased, which may reduce disinfection time.

In light of the disclosure set forth herein, and without limiting the disclosure in any way, in a first aspect of the present disclosure, which may be combined with any other aspect, or portion thereof, a peritoneal dialysis (“PD”) system includes a PD machine including a housing, a PD fluid pump housed by the housing, a plurality of PD fluid lines, and a plurality of PD fluid line connectors positioned and arranged at the housing to accept distal ends of the PD fluid lines to perform a disinfection sequence; and a disinfection unit including a disinfection unit housing, a PD fluid line connector positioned and arranged at the disinfection unit housing for receiving one of the PD fluid lines of the PD machine for the disinfection sequence, a line extending from the disinfection unit housing for connecting to one of the PD fluid line connectors of the PD machine for the disinfection sequence, and a disinfection fluid pump housed by the disinfection unit for pumping disinfection fluid during the disinfection sequence.

In a second aspect of the present disclosure, which may be combined with any other aspect, or portion thereof, the PD fluid pump is set to a free-flow position during the disinfection sequence.

In a third aspect of the present disclosure, which may be combined with any other aspect, or portion thereof, the PD fluid pump is operated at a slow speed during disinfection.

In a fourth aspect of the present disclosure, which may be combined with any other aspect, or portion thereof, the disinfection unit includes a control unit configured to cause the disinfection fluid pump to pump the disinfection fluid during the disinfection sequence at 200 ml/min to 500 ml/min.

In a fifth aspect of the present disclosure, which may be combined with any other aspect, or portion thereof, the disinfection unit includes at least one pressure sensor positioned and arranged to sense a pressure of the disinfection fluid during the disinfection sequence, the at least one pressure sensor having a pressure sensor output to the disinfection unit control unit, and wherein the disinfection unit control unit is configured to use the at least one pressure sensor output as feedback for operating the disinfection fluid pump so that the disinfection fluid is at a desired pressure or within a desired pressure range during the disinfection sequence.

In a sixth aspect of the present disclosure, which may be combined with any other aspect, or portion thereof, the PD machine includes a PD machine control unit in data communication with the disinfection unit control unit, the PD machine further including at least one pressure sensor positioned and arranged to sense a pressure of the disinfection fluid in the PD machine during the disinfection sequence, the at least one pressure sensor having a pressure sensor output delivered via the data communication to the disinfection unit control unit, and wherein the disinfection unit control unit is configured to use the at least one pressure sensor output as feedback for operating the disinfection fluid pump so that the disinfection fluid is at a desired pressure or within a desired pressure range during the disinfection sequence.

In a seventh aspect of the present disclosure, which may be combined with any other aspect, or portion thereof, the disinfection unit includes a control unit and a heater under control of the disinfection unit control unit to cause the disinfection fluid heater to heat the disinfection fluid during the disinfection sequence to 70° C. to 110° C.

In an eighth aspect of the present disclosure, which may be combined with any other aspect, or portion thereof, the disinfection unit includes at least one temperature sensor positioned and arranged to sense a temperature of the disinfection fluid during the disinfection sequence, the at least one temperature sensor having a temperature sensor output to the disinfection unit control unit, and wherein the disinfection unit control unit is configured to use the at least one temperature sensor output as feedback for operating the disinfection fluid pump so that the disinfection fluid is at a desired temperature or within a desired temperature range during the disinfection sequence.

In a ninth aspect of the present disclosure, which may be combined with any other aspect, or portion thereof, the PD machine includes a PD machine control unit in data communication with the disinfection unit control unit, the PD machine further including at least one temperature sensor positioned and arranged to sense a temperature of the disinfection fluid in the PD machine during the disinfection sequence, the at least one temperature sensor having a temperature sensor output delivered via the data communication to the disinfection unit control unit, and wherein the disinfection unit control unit is configured to use the at least one temperature sensor output as feedback for operating the disinfection fluid pump so that the disinfection fluid is at a desired temperature or a desired temperature range during the disinfection sequence.

In a tenth aspect of the present disclosure, which may be combined with any other aspect, or portion thereof, the PD machine includes a heater, and wherein the PD machine heater is not powered during the disinfection sequence.

In an eleventh aspect of the present disclosure, which may be combined with any other aspect, or portion thereof, the PD machine includes a heater, and wherein the PD machine heater is provided with a low amount of power during the disinfection sequence.

In a twelfth aspect of the present disclosure, which may be combined with any other aspect, or portion thereof, the disinfection unit includes a switch configured such that a manual actuation of the switch causes the disinfection fluid pump to begin pumping the disinfection fluid.

In a thirteenth aspect of the present disclosure, which may be combined with any other aspect, or portion thereof, the PD machine includes a PD machine control unit and the disinfection unit includes a disinfection unit control unit in data communication with the PD machine control unit, and wherein the PD machine control unit is configured to automatically command the disinfection unit control unit to cause the disinfection fluid pump to begin pumping the disinfection fluid.

In a fourteenth aspect of the present disclosure, which may be combined with any other aspect, or portion thereof, the PD machine control unit is configured to automatically command the disinfection unit control unit to cause the disinfection fluid pump to begin pumping the disinfection fluid after receiving confirmation that plural disinfection sequence steps have been completed.

In a fifteenth aspect of the present disclosure, which may be combined with any other aspect, or portion thereof, a peritoneal dialysis (“PD”) system includes a PD machine including a housing, a PD fluid pump housed by the housing, a patient line in fluid communication with the PD fluid pump, a patient line connector positioned and arranged at the housing to accept a distal end of the patient line to perform a disinfection sequence, a plurality of PD fluid lines, and a plurality of PD fluid line connectors positioned and arranged at the housing to accept distal ends of the PD fluid lines to perform the disinfection sequence; and a disinfection unit including a disinfection unit housing, a PD fluid line connector positioned and arranged at the disinfection unit housing for receiving one of the PD fluid lines of the PD machine for the disinfection sequence, a line extending from the disinfection unit housing for connecting to one of the PD fluid line connectors of the PD machine for the disinfection sequence, and a disinfection fluid pump housed by the disinfection unit for pumping disinfection fluid during the disinfection sequence.

In a sixteenth aspect of the present disclosure, which may be combined with any other aspect, or portion thereof, the patient line is a dual lumen patient line including a fresh PD fluid lumen and a used PD fluid lumen.

In a seventeenth aspect of the present disclosure, which may be combined with any other aspect, or portion thereof, at least one of the patient line, the plurality of PD fluid lines or the line extending from the disinfection unit housing is a flexible line.

In an eighteenth aspect of the present disclosure, which may be combined with any other aspect, or portion thereof, a peritoneal dialysis (“PD”) system includes a PD machine including a PD machine control unit, a PD fluid pump under control of the PD machine control unit; and a disinfection unit configured to be placed in disinfection fluid communication with the PD machine, the disinfection unit including a disinfection unit control unit configured to be placed in data communication with the PD machine control unit, and a disinfection fluid pump under control of the disinfection unit control unit, wherein the PD machine control unit is configured to automatically command the disinfection unit control unit to cause the disinfection fluid pump to begin pumping disinfection fluid in a disinfection sequence.

In a nineteenth aspect of the present disclosure, which may be combined with any other aspect, or portion thereof, the PD machine control unit is configured to automatically command the disinfection unit control unit to cause the disinfection fluid pump to begin pumping the disinfection fluid after receiving confirmation that plural disinfection sequence steps have been completed.

In a twentieth aspect of the present disclosure, which may be combined with any other aspect, or portion thereof, any of the features, functionality and alternatives described in connection with any one or more of FIGS. 1 and 2 may be combined with any of the features, functionality and alternatives described in connection with any other of FIGS. 1 and 2.

In light of the above aspects and present disclosure set forth herein, it is an advantage of the present disclosure to provide a durable peritoneal dialysis (“PD”) system using disinfection, where a disinfection fluid flowrate and/or temperature is increased.

It is another advantage of the present disclosure to provide a durable peritoneal dialysis (“PD”) system using disinfection, where a disinfection sequence efficiency is increased.

It is a further advantage of the present disclosure to provide a durable peritoneal dialysis (“PD”) system using disinfection, where component wear is reduced.

It is yet another advantage of the present disclosure to provide a durable peritoneal dialysis (“PD”) system using disinfection, which is relatively easy to implement.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the improvements or advantages listed herein, and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system in a treatment state.

FIG. 2 is a fluid flow schematic of one embodiment for a medical fluid, e.g., PD fluid, system in a disinfection state using a disinfection unit of the present disclosure.

### DETAILED DESCRIPTION

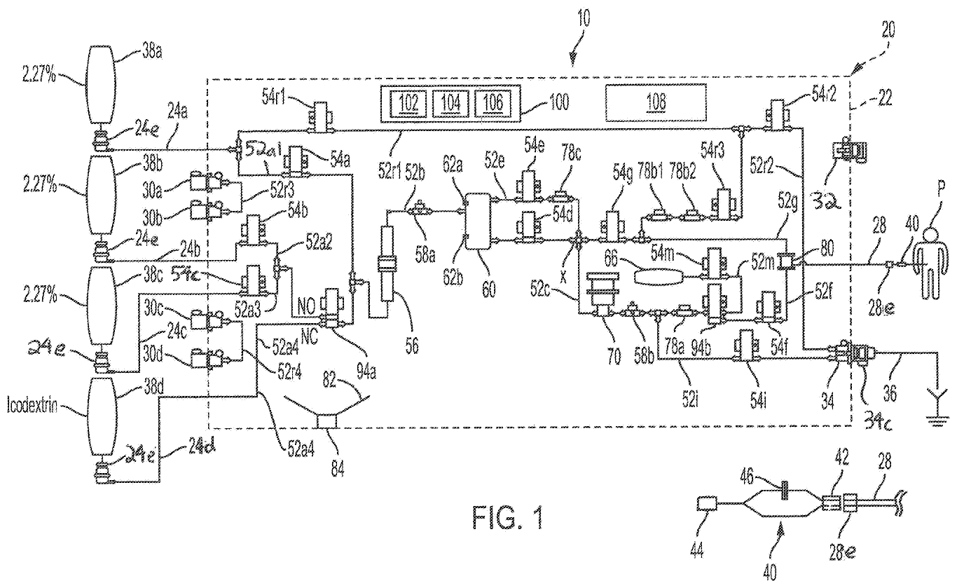

Referring now to the drawings and in particular to FIG. 1, a medical system having the enhanced disinfection of the present disclosure is illustrated via peritoneal dialysis (“PD”) system 10. System 10 includes a PD machine or cycler 20 and a control unit 100 having one or more processor 102, one or more memory 104, video controller 106 and user interface 108. User interface 108 may alternatively or additionally be a remote user interface, e.g., via a tablet or smartphone. Control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. The transceiver is also used for communication with a disinfection unit 110 as discussed herein. Control unit 100 in an embodiment controls all electrical fluid flow and heating components of PD machine or cycler 20 and receives outputs from all sensors of the PD machine. System 10 in the illustrated embodiment includes durable and reusable components that contact fresh and used PD fluid, which necessitates that PD machine or cycler 20 be disinfected between treatments, e.g., via heat disinfection.

System 10 in FIG. 1 includes an inline resistive heater 56, reusable supply lines or tubes 52a1 to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a and 58b, pressure sensors 78a, 78b1, 78b2 and 78c, reusable patient tubing or lines 52f and 52g having respective valves 54f and 54g, a dual lumen patient line 28, a hose reel 80 for retracting patient line 28, reusable drain tubing or line 52i extending to drain line connector 34 and having a drain line valve 54i, and reusable recirculation disinfection tubing or lines 52r1 and 52r2 operating with respective disinfection valves 54r1 and 54r2. A third recirculation or disinfection tubing or line 52r3 extends between disinfection or PD fluid line connectors 30a and 30b for use during disinfection. A fourth recirculation or disinfection tubing or line 52r4 extends between disinfection connectors 30c and 30d for use during disinfection.

System 10 further includes PD fluid containers or bags 38a to 38c (e.g., holding the same or different formulations of PD fluid), which connect to distal ends 24e of reusable PD fluid lines 24a to 24c, respectively. System 10d further includes a fourth PD fluid container or bag 38d that connects to a distal end 24e of reusable PD fluid line 24d. Fourth PD fluid container or bag 38d may hold the same or different type (e.g., icodextrin) of PD fluid than provided in PD fluid containers or bags 38a to 38c. Reusable PD fluid lines 24a to 24d extend in one embodiment through apertures (not illustrated) defined or provided by housing 22 of PD machine or cycler 20.

System 10 in the illustrated embodiment includes four disinfection or PD fluid line connectors 30a to 30d for connecting to distal ends 24e of reusable PD fluid lines 24a to 24d, respectively, during disinfection. System 10 also provides a patient line connector 32 that includes an internal lumen, e.g., a U-shaped lumen, which for disinfection directs fresh PD fluid from one PD fluid lumen of a connected distal end 28e of dual lumen patient line 28 into the other PD fluid lumen. Reusable supply tubing or lines 52a1 to 52a4 communicate with reusable supply lines 24a to 24d, respectively. Reusable supply tubing or lines 52a1 to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) 2.27% (or other) glucose dialysis fluid from container or bag 38b or 38c and (ii) icodextrin from container or bag 38d. In the illustrated embodiment, icodextrin from container or bag 38d is connected to the normally closed port of three-way valve 94a.

System 10 is constructed in one embodiment such that drain line 52i during a patient fill is fluidly connected downstream from PD fluid pump 70. In this manner, if drain valve 54i fails or somehow leaks during the patient fill of patient P, fresh PD fluid is pushed down disposable drain line 36 instead of used PD fluid potentially being pulled into pump 70. Disposable drain line 36 is in one embodiment removed for disinfection, wherein drain line connector 34 is capped via a cap 34c to form a closed disinfection loop. PD fluid pump 70 may be an inherently accurate pump, such as a piston pump, or less accurate pump, such as a gear pump that operates in cooperation with a flowmeter (not illustrated) to control fresh and used PD fluid flowrate and volume.

System 10 may further include a leak detection pan 82 located at the bottom of housing 22 of cycler 20 and a corresponding leak detection sensor 84 outputting to control unit 100. In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along vent line 52e, which may be filled with air or a mixture of air and PD fluid, but which should nevertheless be at the same negative pressure as PD fluid located within PD fluid line 52c.

System 10 in the example of FIG. 1 includes redundant pressure sensors 78b1 and 78b2, the output of one of which is used for pump control, while the output of the other pressure sensor is a safety or watchdog output to make sure the control pressure sensor is reading accurately. Pressure sensors 78b1 and 78b2 are located along a line including a third recirculation valve 54r3. System 10 may further employ one or more cross, marked via an X in FIG. 1, which may (i) reduce the overall amount and volume of the internal, reusable tubing, (ii) reduce the number of valves needed, and (iii) allow the portion of the fluid circuitry shared by both fresh and used PD fluid to be minimized.

System 10 in the example of FIG. 1 further includes a source of acid, such as a citric acid container or bag 66. Citric acid container or bag 66 is in selective fluid communication with second three-way valve 94b via a citric acid valve 54m located along a citric acid line 52m. Citric acid line 52m is connected in one embodiment to the normally closed port of second three-way valve 94b, so as to provide redundant valves between citric acid container or bag 66 and the PD fluid circuit during treatment. The redundant valves ensure that no citric (or other) acid reaches the treatment fluid lines during treatment. Citric (or other) acid is used instead during disinfection.

Control unit 100 in an embodiment uses feedback from any one or more of pressure sensors 78a to 78c to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, between −5 kPa (−0.73 psig) and −15 kPa (−2.2 psig), such as −9 kPa (−1.3 psig) or higher (more negative). The pressure feedback may be used in a proportional, integral, derivative (“PID”) pressure routine for pumping fresh and used PD fluid at a desired positive or negative pressure.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature.

FIG. 1 also illustrates that system 10 includes and uses a disposable filter set 40, which communicates fluidly with the fresh and used PD fluid lumens of dual lumen patient line 28. Disposable filter set 40 includes a disposable connector 42 that connects to a distal end 28e of reusable patient line 28. Disposable filter set 40 also includes a connector 44 that connects to the patient's transfer set. Disposable filter set 40 further includes a hydrophilic filter membrane 46, which may be a sterilizing grade filter, which further filters fresh PD fluid. Disposable filter set 40 is provided in one embodiment as a last chance filter for PD machine 20, which has been heat disinfected between treatments. Any pathogens that may remain after disinfection, albeit unlikely, are filtered from the PD fluid via the hydrophilic filter membrane 46 of disposable filter set 40.

FIG. 1 illustrates system 10 setup for treatment with PD fluid containers or bags 38a to 38d connected via reusable, flexible PD fluid lines 24a to 24d, respectively. Dual lumen patient line 28 is connected to patient P via disposable filter set 40. Disposable drain line 36 is connected to drain line connector 34. In FIG. 1, PD machine or cycler 20 of system 10 is configured to perform multiple patient drains, patient fills, patient dwells, and a priming procedure, as part of or in preparation for treatment.

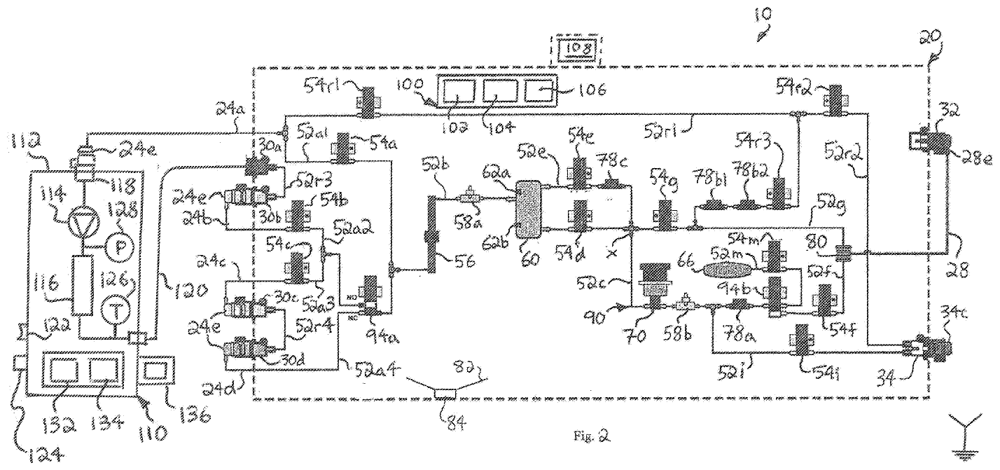

FIG. 2 illustrates system 10 in a disinfection mode. Three of four PD fluid containers or bags 38a to 38d are removed and the corresponding flexible PD fluid lines 24a to 24d are plugged instead in a sealed manner into the corresponding disinfection or PD fluid line connectors 30a to 30d, respectively. Reusable dual lumen patient line 28 is disconnected from disposable filter set 40 (which is discarded), and distal end 28e of dual lumen patient line 28 is plugged sealingly into patient line connector 32. Disposable drain line 36 is removed from drain line connector 34 and discarded. Drain line connector 34 is capped via cap 34c to form a closed disinfection loop 90. PD machine or cycler 20 of system 10 in FIG. 2 is configured to perform a disinfection sequence, e.g., a heat disinfection sequence in which fresh PD fluid is heated to a disinfection temperature, e.g., 70° C. to 90° C., or higher as discussed herein. The heated PD fluid is circulated within closed disinfection loop 90 for an amount of time needed to properly disinfect the fluid components and lines of the disinfection loop.

To provide good disinfection and heat transfer to each of the lines disinfected, PD fluid pump 70 if used as the primary distinction sequence pump would need to run at a relatively high pumping rate, resulting in risk of cavitation and the wear of the associated pump seals. It is accordingly contemplated not to use PD fluid pump 70, either at all, or as the primary disinfection fluid driving pump during the disinfection sequence.

FIG. 2 illustrates that system 10 of the present disclosure instead provides a disinfection unit 110, which may be separate from PD machine or cycler 20. Disinfection unit 110 includes a disinfection unit housing 112 holding an additional pump 114, which may be used in addition to or instead of PD fluid pump 70 during disinfection. To that end, disinfection unit pump 114 may be a sturdy pump that is less accurate than PD fluid pump 70, but which may operate at higher flowrates than PD fluid pump 70 without incurring appreciable wear. Disinfection unit pump 114 may for example be a centrifugal pump. The higher flowrate may for example be from 200 milliliters (“ml”)/min to 500 ml/min. FIG. 2 further illustrates that disinfection unit 110 optionally includes a second, disinfection unit inline heater 116 located within disinfection unit housing 112, which is selected specifically to heat fluid moving at the elevated disinfection flowrates and to heat the disinfection fluid to a temperature specified herein or to a potentially higher disinfection temperature, such as 90° C. to 110° C.

Disinfection unit 110 in one embodiment includes a PD fluid line connector 118 at disinfection unit housing 112 that accepts a distal end 24e of any desired one of the reusable, flexible PD fluid lines 24a to 24d of PD fluid machine or cycler 20. In the illustrated embodiment, PD fluid line connector 118 accepts distal end 24e of reusable, flexible PD fluid line 24a. Disinfection unit 110 also includes a flexible line 120 that extends from disinfection unit housing 112 to the open PD fluid line connector 30a to 30d located at PD fluid machine or cycler 20. In the illustrated embodiment, flexible line 120 is connected to PD fluid line connector 30a. The two fluid connections to disinfection unit 110 enable the internal lines and fluid components of the disinfection unit to form part of the PD machine or cycler's disinfection loop 90 during the disinfection sequence. Sealed connections to PD fluid line connector 118 and flexible line 120 may be made by the patient or caregiver after treatment when it is time for disinfection.

Electrical control may be implemented in disinfection unit 110 in a variety of different ways. If only disinfection unit pump 114 is provided, then an on/off button or switch 122 may be provided at disinfection unit housing 112, which the patient or caregiver may switch to “on” to initiate a disinfection sequence. Disinfection unit 110 includes an alternating current (“AC”) plug 124 at disinfection unit housing 112 and whatever electronics (within housing 112) are necessary to convert the AC voltage to the disinfection unit pump voltage if necessary. If a disinfection unit heater 116 is also provided, on/off switch 122 may still be provided for the patient or caregiver, however, at least one temperature sensor 126 and a disinfection unit control unit 130 for using an output from one or more temperature sensor 126 as feedback for controlling power to inline heater 116 are provided so that the disinfection fluid, which may be PD fluid, exits disinfection unit 110 to PD machine or cycler 20 at a desired disinfection temperature or within a desired temperature range.

When needed, disinfection unit 110 may also include one or more pressure sensor 128. The disinfection unit control unit 130 uses an output from the one or more pressure sensor 128 as feedback for controlling disinfection unit pump 114, so that the disinfection fluid exits the disinfection unit to the PD machine or cycler at a desired disinfection pressure or within a desired pressure range. Disinfection unit control unit 130 in the illustrated embodiment may be housed within disinfection unit hosing 112 and include one or more processor 132 and one or more memory 134. Disinfection unit control unit 130 may also include a user interface 136, e.g., a single or multi-line light-emitting diode (“LED”) or liquid crystal display (“LCD”), wherein, the control unit may further include a video controller.

As discussed herein, control unit 100 of PD machine or cycler 20 may also include a transceiver and a wired or wireless connection to a network (not illustrated), e.g., the internet, etc. Control unit 130 of disinfection unit 110 may likewise include a transceiver for wireless communication with control unit 100 of PD machine or cycler 20, e.g., via Bluetooth or Wifi wireless technologies. Alternatively or additionally, control unit 130 may be placed in wired communication with control unit 100, e.g., via an ethernet cable. In an embodiment, instead of the patient or caregiver powering disinfection unit 110 at the time of disinfection, control unit 100 of PD machine or cycler 20 tells control unit 130 of disinfection unit 110 when to power disinfection unit pump 114 and disinfection unit heater 116 if provided, e.g., after control unit 100 of PD machine or cycler 20 has confirmed that the patient or caregiver has made all necessary connections for the disinfection sequence. The patient or caregiver here may accordingly connect disinfection unit 110 to PD machine or cycler 20 as described herein, make all other necessary disinfection connections, and then be free to leave and perform other tasks.

It is also contemplated to wirelessly transmit the outputs from one or more temperature sensor 58a, 5bb and/or one or more pressure sensor 78a, 78b1, 78b2, 78c of PD machine or cycler 20 to control unit 130 of disinfection unit 110 for use as feedback to control, respectively, the power applied to disinfection unit heater 116 and/or the rate (or other pressure adjusting parameter) of disinfection unit pump 114. In this manner, disinfection fluid, e.g., PD fluid, may be delivered from disinfection unit 110 at the desired disinfection temperature and/or pressure without having to provide corresponding temperature and/or pressure sensors within the disinfection unit.

During disinfection, control unit 100 of PD machine or cycler 20 is programmed in one embodiment to set PD fluid pump 70, which may be a piston pump, such that it is in a free-flow position where the disinfection fluid may flow through the PD fluid pump without operating the pump. In an alternative embodiment, control unit 100 operates PD fluid pump at a slow or reduced speed during disinfection. Likewise, control unit 100 of PD machine or cycler 20 is programmed in one embodiment to not power inline heater 58 of PD machine or cycler 20 during disinfection if disinfection unit 110 is provided with its own heater 58. In an alternative embodiment, control unit 100 provides a low amount of power to inline heater 58 of PD machine or cycler 20 during disinfection even if disinfection unit 110 is provided with its own heater 116. Control unit 100 may provide full power to inline heater 58 of PD machine or cycler 20 during disinfection if disinfection unit 110 is not provided with its own heater. Here, inline heater 58 of PD machine or cycler 20 is relied upon to heat the disinfection fluid to the desired disinfection temperature.

After disinfection, the disinfection unit 110 may be left connected to PD machine or cycler 20 until it is time for the next treatment. At that time, the patient or caregiver may (i) disconnect the disinfection unit's flexible line 120 from the PD fluid line connector (e.g., connector 30a) located at PD fluid machine or cycler 20, (ii) disconnect distal end 28e of the reusable, flexible PD fluid line (e.g., line 24a) of PD fluid machine or cycler 20 from the PD fluid line connector 118 of disinfection unit 110, and (iii) connect the disinfection unit's flexible line 120 to the disinfection unit's PD fluid line connector 118. Such disconnection and reconnection closes disinfection unit 110 fluidically and maintains it in a relatively clean and disinfected state. Disinfection unit 110 is then ready for its next use.

It is contemplated that disinfection unit 110 is relatively small, lightweight and easy to transport to and from PD machine or cycler 20 as needed if it is desired to do so (or could be left sitting next to PD machine or cycler 20). Housing 112 may therefore be provided with a handle for carrying disinfection unit 110. Housing 112 may also be provided with feet for setting disinfection unit 110 next to PD machine or cycler 20. Housing 112 may also be provided with wheels or casters if needed.

It should be understood that various changes and modifications to the presently preferred embodiments described herein will be apparent to those skilled in the art. It is therefore intended that such changes and modifications be covered by the appended claims. For example, while disinfection unit 110 has been described herein as a unit separate from PD machine or cycler 20, some or all of the components of disinfection unit 110 may be integrated into PD machine or cycler 20 or be packaged in a housing that is merged with housing 22 of PD machine or cycler 20.

The invention is claimed as follows:

1. A peritoneal dialysis (“PD”) system comprising:
a PD machine including
a housing,
a PD fluid pump housed by the housing,
a plurality of PD fluid lines, and
a plurality of PD fluid line connectors positioned and arranged at the housing to accept distal ends of the PD fluid lines to perform a disinfection sequence; and
a disinfection unit including
a disinfection unit housing,
a PD fluid line connector positioned and arranged at the disinfection unit housing for receiving one of the PD fluid lines of the PD machine for the disinfection sequence,
a line extending from the disinfection unit housing for connecting to one of the PD fluid line connectors of the PD machine for the disinfection sequence, and
a disinfection fluid pump housed by the disinfection unit for pumping disinfection fluid during the disinfection sequence.

2. The PD system of claim 1, wherein the PD fluid pump is set to a free-flow position during the disinfection sequence.

3. The PD system of claim 1, wherein the PD fluid pump is operated at a slow speed during disinfection.

4. The PD system of claim 1, wherein the disinfection unit includes a control unit configured to cause the disinfection fluid pump to pump the disinfection fluid during the disinfection sequence at 200 ml/min to 500 ml/min.

5. The PD system of claim 4, wherein the disinfection unit includes at least one pressure sensor positioned and arranged to sense a pressure of the disinfection fluid during the disinfection sequence, the at least one pressure sensor having a pressure sensor output to the disinfection unit control unit, and wherein the disinfection unit control unit is configured to use the at least one pressure sensor output as feedback for operating the disinfection fluid pump so that the disinfection fluid is at a desired pressure or within a desired pressure range during the disinfection sequence.

6. The PD system of claim 4, wherein the PD machine includes a PD machine control unit in data communication with the disinfection unit control unit, the PD machine further including at least one pressure sensor positioned and arranged to sense a pressure of the disinfection fluid in the PD machine during the disinfection sequence, the at least one pressure sensor having a pressure sensor output delivered via the data communication to the disinfection unit control unit, and wherein the disinfection unit control unit is configured to use the at least one pressure sensor output as feedback for operating the disinfection fluid pump so that the disinfection fluid is at a desired pressure or within a desired pressure range during the disinfection sequence.

7. The PD system of claim 1, wherein the disinfection unit includes a control unit and a heater under control of the disinfection unit control unit to cause the disinfection fluid heater to heat the disinfection fluid during the disinfection sequence to 70° C. to 110° C.

8. The PD system of claim 7, wherein the disinfection unit includes at least one temperature sensor positioned and arranged to sense a temperature of the disinfection fluid during the disinfection sequence, the at least one temperature sensor having a temperature sensor output to the disinfection unit control unit, and wherein the disinfection unit control unit is configured to use the at least one temperature sensor output as feedback for operating the disinfection fluid pump so that the disinfection fluid is at a desired temperature or within a desired temperature range during the disinfection sequence.

9. The PD system of claim 7, wherein the PD machine includes a PD machine control unit in data communication with the disinfection unit control unit, the PD machine further including at least one temperature sensor positioned and arranged to sense a temperature of the disinfection fluid in the PD machine during the disinfection sequence, the at least one temperature sensor having a temperature sensor output delivered via the data communication to the disinfection unit control unit, and wherein the disinfection unit control unit is configured to use the at least one temperature sensor output as feedback for operating the disinfection fluid pump so that the disinfection fluid is at a desired temperature or a desired temperature range during the disinfection sequence.

10. The PD system of claim 7, wherein the PD machine includes a heater, and wherein the PD machine heater is not powered during the disinfection sequence.

11. The PD system of claim 7, wherein the PD machine includes a heater, and wherein the PD machine heater is provided with a low amount of power during the disinfection sequence.

12. The PD system of claim 1, wherein the disinfection unit includes a switch configured such that a manual actuation of the switch causes the disinfection fluid pump to begin pumping the disinfection fluid.

13. The PD system of claim 1, wherein the PD machine includes a PD machine control unit and the disinfection unit includes a disinfection unit control unit in data communication with the PD machine control unit, and wherein the PD machine control unit is configured to automatically command the disinfection unit control unit to cause the disinfection fluid pump to begin pumping the disinfection fluid.

14. The PD system of claim 13, wherein the PD machine control unit is configured to automatically command the disinfection unit control unit to cause the disinfection fluid pump to begin pumping the disinfection fluid after receiving confirmation that plural disinfection sequence steps have been completed.

15. A peritoneal dialysis (“PD”) system comprising:
a PD machine including
a housing,
a PD fluid pump housed by the housing,
a patient line in fluid communication with the PD fluid pump,
a patient line connector positioned and arranged at the housing to accept a distal end of the patient line to perform a disinfection sequence,
a plurality of PD fluid lines, and
a plurality of PD fluid line connectors positioned and arranged at the housing to accept distal ends of the PD fluid lines to perform the disinfection sequence; and
a disinfection unit including
a disinfection unit housing,
a PD fluid line connector positioned and arranged at the disinfection unit housing for receiving one of the PD fluid lines of the PD machine for the disinfection sequence,
a line extending from the disinfection unit housing for connecting to one of the PD fluid line connectors of the PD machine for the disinfection sequence, and
a disinfection fluid pump housed by the disinfection unit for pumping disinfection fluid during the disinfection sequence.

16. The PD system of claim 15, wherein the patient line is a dual lumen patient line including a fresh PD fluid lumen and a used PD fluid lumen.

17. The PD system of claim 15, wherein at least one of the patient line, the plurality of PD fluid lines or the line extending from the disinfection unit housing is a flexible line.

18. A peritoneal dialysis (“PD”) system comprising:
a PD machine including
a PD machine control unit,
a PD fluid pump under control of the PD machine control unit; and
a disinfection unit configured to be placed in disinfection fluid communication with the PD machine, the disinfection unit including
a disinfection unit control unit configured to be placed in data communication with the PD machine control unit, and
a disinfection fluid pump under control of the disinfection unit control unit,
wherein the PD machine control unit is configured to automatically command the disinfection unit control unit to cause the disinfection fluid pump to begin pumping disinfection fluid in a disinfection sequence.

19. The PD system of claim 18, wherein the PD machine control unit is configured to automatically command the disinfection unit control unit to cause the disinfection fluid pump to begin pumping the disinfection fluid after receiving confirmation that plural disinfection sequence steps have been completed.

20. The PD system of claim 18, wherein the disinfection unit includes a switch configured such that an actuation of the switch causes the disinfection fluid pump to begin pumping the disinfection fluid.
